(19) United States
(12) Patent Application Publication
(10) Pub. No.: US 2025/0283885 A1
(43) Pub. Date: Sep. 11, 2025

(54) MODULAR BIOSENSOR FOR RECEPTOR TYROSINE KINASE ACTIVITY

(71) Applicant: The Trustees of Princeton University, Princeton, NJ (US)

(72) Inventors: Jared Toettcher, Princeton, NJ (US); Caleb Bashor, Houston, TX (US); Celeste Nelson, Princeton, NJ (US); Payam Farahani, Seattle, WA (US)

(73) Assignee: The Trustees of Princeton University, Princeton, NJ (US)

(21) Appl. No.: 18/804,650

(22) Filed: Aug. 14, 2024

Related U.S. Application Data
(60) Provisional application No. 63/562,572, filed on Mar. 7, 2024.

(51) Int. Cl. G01N 33/573; G01N 33/554; G01N 33/68

(52) U.S. Cl. CPC G01N 33/573; G01N 33/554; G01N 33/6872; G01N 2333/71; G01N 2333/912

(57) ABSTRACT

A modular biosensor (pYtags) for receptor tyrosine kinase activity may be provided, along with systems and techniques incorporating such biosensors. pYtags may include a cell surface receptor, such as a receptor tyrosine kinase (RTK), modified with a tyrosine activation motif that, when phosphorylated, recruits, e.g., a fluorescently labeled tandem SH2 domain with high specificity. Orthogonal pYtags can be used to monitor the dynamics of, e.g., EGFR and ErbB2 activity in the same cell, revealing distinct phases of activation for each RTK.

### CROSS-REFERENCE TO RELATED APPLICATIONS

The present application claims priority to U.S. Provisional Patent Application No. 63/532,572, filed Aug. 14, 2023, the contents of which are incorporated by reference herein in its entirety.

### STATEMENT REGARDING FEDERALLY SPONSORED RESEARCH OR DEVELOPMENT

This invention was made with government support under Grant Nos. 1750663 and 2134935 awarded by the National Science Foundation, Grant Nos. HL164861, HD111539, GM007388, and EB032272 awarded by the National Institutes of Health, and Grant No. N00014-21-1-4006 awarded by the Office of Naval Research. The government has certain rights in the invention.

### TECHNICAL FIELD

The present disclosure relates to biosensors for receptor tyrosine kinases (RTKs), and specifically to a modular approach for using such biosensors to, e.g., monitor the activity of a user-defined RTK by live-cell microscopy.

### BACKGROUND

RTKs are major signaling hubs in metazoans, playing crucial roles in cell proliferation, migration, and differentiation. However, few tools are available to measure the activity of a specific RTK in individual living cells.

### BRIEF SUMMARY

In various aspects, an engineered biosensor may be provided. The biosensor may be an amino acid-based biosensor. The biosensor may be useful for, e.g., monitoring receptor activity. The biosensor may include a cell surface receptor and a tyrosine residue coupled to the cell surface receptor, where the tyrosine residue is configured to be phosphorylated by the cell surface receptor when the cell surface receptor is activated.

In certain aspects, the cell surface receptor may be a receptor tyrosine kinase. In certain aspects, the RTK may be an epidermal growth factor receptor (EGFR). In certain aspects, the RTK may be a fibroblast growth factor receptor (FGFR). In certain aspects, the RTK may be a platelet-derived growth factor receptor (PDGFR). In certain aspects, the RTK may be a vascular endothelial growth factor receptor (VEGFR).

In various aspects, a system may be provided. The system may include a first engineered biosensor as disclosed herein, and a reporter protein. The reporter protein may include two parts. The first part may be configured to specifically interact with a phosphorylated tyrosine residue of the first biosensor. The second part may be, e.g., a fluorescent protein, a protein domain that triggers an intracellular signaling response, or a combination thereof. The first part may be a structurally conserved protein domain. The structurally conserved protein domain may be a Src Homology 2 (SH2) domain. The system may include a second engineered biosensor as disclosed herein, where the second biosensor may be orthogonal to the first biosensor.

In various aspects, an engineered biosensor sequence may be provided. The biosensor sequence may include a first polynucleotide sequence encoding a cell surface receptor, and a second polynucleotide sequence fused to the first polynucleotide sequence, the second polynucleotide sequence encoding a tyrosine residue that can be phosphorylated by the cell surface receptor.

In various aspects, a method for monitoring receptor tyrosine kinase activity may be provided. The method may include measuring a first luminescence of at least one cell, the at least one cell having a cell surface. The method may include allowing a cell surface receptor of an engineered biosensor as disclosed herein to interact with the cell surface of the at least one cell and activate, convert a tyrosine residue of the biosensor into a phosphorylated tyrosine residue, and recruit a reporter protein to the phosphorylated tyrosine residue, where the reporter protein includes a fluorescent protein. The method may then include measuring a second luminescence of the at least one cell. As disclosed herein, the cell surface receptor may be, e.g., an EGFR, a FGFR, a PDGFR, a VEGFR, etc. The method may include continuously measuring luminescence or fluorescence of the at least one cell over a period of time. The method may include determining a difference in luminescence or fluorescence of the at least one cell, after exposing the at least one cell to a pharmaceutical agent. The reporter protein may include a structurally conserved protein domain fused to a fluorescent protein, and optionally a protein domain that triggers an intracellular signaling response. As disclosed herein, the structurally conserved protein domain may be, e.g., a Src Homology 2 (SH2) domain, and is preferably a tandem domain.

### BRIEF DESCRIPTION OF DRAWINGS

FIG. 1 is a schematic illustration of a first embodiment of a phosphotyrosine tag (pYtag) system.

FIGS. 2A and 2B are schematic illustrations of a first embodiment of the pYtag system used as part of a localization-based signaling readout, with low RTK activity (2A) and high RTK activity (2B).

FIG. 3 is a schematic illustration of a second embodiment of a pYtag system.

FIGS. 4A and 4B are schematic illustrations of a second embodiment of a pYtag system used as part of a localization-based signaling readout, with low RTK activity (4A) and high RTK activity (4B).

FIG. 5A is a set of representative images of NIH 3T3 and SYF cells expressing EGFR pYtag, treated with EGF (100 ng/mL); Scale bars represent 40 μm.

FIG. 5B is a plot showing mean clearance of cytosolic ZtSH2 in SYF and NIH 3T3 cells 10 min after treatment with EGF; for each condition, n>20 cells from 2 independent experiments.

FIG. 6 is a plot showing mean±SD clearance of cytosolic ZtSH2 from EGFR pYtag-expressing NIH3T3 cells treated with EGF (20 ng/ml).

FIG. 7 is a schematic showing MCF10A human mammary epithelial cells cultured on soft substrata form round, multilayered clusters. EGFR pYtag and ErkKTR were used to spatiotemporally monitor both EGFR and Erk responses after stimulation with EGF.

FIG. 8 shows images of MCF10A cells cultured on soft substrata and treated with EGF (100 ng/mL); scale bar represents 25 um.

FIGS. 9A, 9B, and 9C are plots showing mean responses of EGFR pYtag-expressing NIH 3T3 cells to varying doses of EGF (9A), epiregulin (EREG) (9B), and epigen (EPGN) (9C); the same 0 ng/mL control was used for each ligand; n=2 independent experiments.

FIG. 10A is a schematic model of EGFR pYtag

FIGS. 10B and 10C are schematic illustrations of how ligand identity may affect signaling, either via ligand-receptor binding (10B) or receptor dimerization (10C).

FIG. 11A is a plot showing predicted mean pYtag responses of WT and GBM-associated mutant EGFRs in NIH 3T3 cells after EREG treatment (20 ng/ml).

FIG. 11B is a plot showing mean actual pYtag response of WT and GBM-associated mutant EGFRs in NIH 3T3 cells after EREG treatment (20 ng/mL); n=3 independent experiments.

FIG. 12A shows representative images of NIH 3T3 cells treated with EGF (100 ng/ml); scale bar represents 20 μm.

FIG. 12B is a plot showing mean clearance of cytosolic ZtSH2 after treatment with EGF (100 ng/mL); n =3 independent experiments.

FIG. 13A is a plot showing mean trajectories for EGFR and ErbB2 activity using multiplexed pYtags; for each reporter, the mean response was normalized to its minimum and maximum measured values. n=3 independent experiments.

FIG. 13B is a plot showing time to half maximal response for individual cells from FIG. 13A; lines denote mean values, boxes denote 25-75th percentiles, and whiskers denote minima and maxima; n>30 cells from 3 independent experiments; ***p<0.001 by Kolmogorov-Smirnov test.

FIG. 14A is a plot showing mean clearance of ZtSH2 from the cytosol following treatment with EGF (100 ng/mL); parental 293T, n=3 independent experiments; knock-in 293T, n=4 independent experiments.

FIG. 14B is a plot showing clearance of ZtSH2 from the cytosol 1 min after treatment with EGF in FIG. 14A; lines denote mean values, boxes denote 25-75th percentiles, and whiskers denote minima and maximal; parental 293T, n =23 cells from 3 independent experiments; knock-in 293T, n=46 cells from 4 independent experiments; ***p<0.001 by Kolmogorov-Smirnov test.

### DETAILED DESCRIPTION

As further detailed in the Appendix, disclosed herein is a modular biosensor for receptor tyrosine kinase activity.

As used herein, the term “fluorescent tag” refers to a molecule having the ability to emit light of a certain wavelength when activated by light of another wavelength. Such “fluorescent tags” may include, but are not limited, to fluorescein, rhodamine, FusionRed, mCherry, GFP, 6-FAM, TET, HEX, Cy5, and Cy3.

As used herein, the term “orthogonal”, when referring to two biosensors, indicates that the two biosensors can operate within a system without interfering with each other.

As used herein, the term “receptor tyrosine kinase” refers to any receptor that recruits tyrosine kinase activity, whether or not it falls under conventional definitions of an RTK.

As used herein, the term “receptor tyrosine kinase” refers to any receptor that recruits tyrosine kinase activity, whether or not it falls under conventional definitions of an RTK.

As used herein, the term “residue” refers to an amino acid that is incorporated into a protein. The amino acid may be a naturally occurring amino acid and, unless otherwise limited, may encompass known analogs of natural amino acids that can function in a similar manner as naturally occurring amino acids.

Here, disclosed is pYtags, a modular approach for monitoring the activity of a user-defined RTK by live-cell microscopy. pYtags comprise an RTK modified with a tyrosine activation motif that, when phosphorylated, can recruit, e.g., a fluorescently labeled tandem SH2 domain with high specificity.

As detailed herein, pYtags enable the monitoring of a specific RTK on seconds-to-minutes time scales and across subcellular and multicellular length scales. Using a pYtag biosensor for epidermal growth factor receptor (EGFR), one can quantitively characterize how signaling dynamics vary with the identity and dose of activating ligand. Further, orthogonal pYtags can be used to monitor the dynamics of, e.g., EGFR and ErbB2 activity in the same cell, revealing distinct phases of activation for each RTK. The specificity and modularity of pYtags opens the door to robust biosensors of multiple tyrosine kinases and may enable engineering of synthetic receptors with orthogonal response programs.

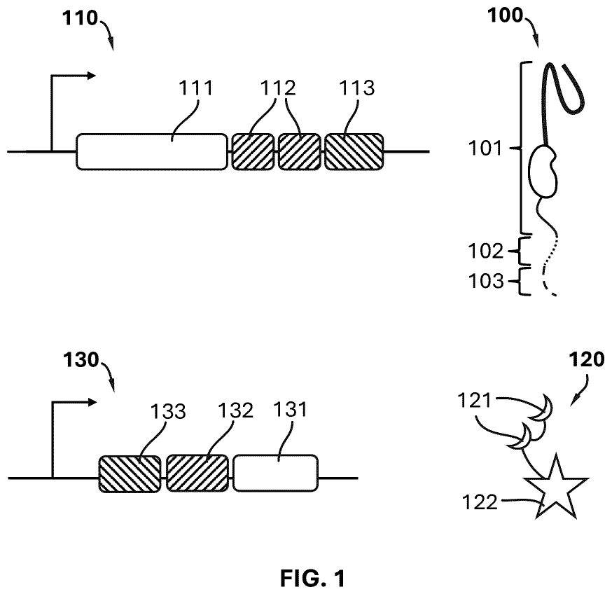

In various aspects, an engineered biosensor may be provided. Referring to FIG. 1, the biosensor (100) may be an amino acid-based biosensor. The biosensor (100) may generally comprise or consist of two parts: a first part (101) including a cell surface receptor (e.g., the RTK) and a second part (102) that includes a tyrosine residue coupled to the cell surface receptor. A third part (103) may optionally be included, which may include, e.g., one or more additional proteins, peptides, etc., such as a reporter tag. In a preferred embodiment, the second part may be appended to the C-terminal tail of the RTK.

In certain aspects, the cell surface receptor may be a receptor tyrosine kinase. In certain aspects, the RTK may be an epidermal growth factor receptor (EGFR). In certain aspects, the RTK may be an epidermal growth factor receptor (EGFR). In certain aspects, the RTK may be a fibroblast growth factor receptor (FGFR). In certain aspects, the RTK may be a platelet-derived growth factor receptor (PDGFR). In certain aspects, the RTK may be a vascular endothelial growth factor receptor (VEGFR). In certain aspects, the RTK may be an insulin receptor. In certain aspects, the RTK may be a rearranged during transfection (RET) receptor. In certain aspects, the RTK may be an erythropoietin-producing human hepatocellular (Eph) receptor. In certain aspects, the RTK may be a hematopoietic stem cell proliferation factor receptor. Members of the protein family of RTKs include, but are not limited to, members of various sub-families, such as the EGFR subfamilies (including ErbB1/EGFR, ErbB2/HER2, ErbB3/HER3, and ErbB4/HER4), the FGFR subfamilies (including FGF1, FGF2, FGF3, FGF4, FGF5, FGF6, FGF7, FGF18, and FGF21), VEGFR subfamilies (including VEGF-A, VEGF-B, VEGF-C, VEGF-D, and PIGF), FMS-like tyrosine kinase (including FLT3), and erythropoietin-producing human hepatocellular (Eph) receptor subfamilies (including EphA1, EphA2, EphA3, EphA4, EphA5, EphA6, EphA7, EphA8, EphA9, EphA10, EphB1, EphB2. EphB3, EphB4, and EphB6).

The tyrosine residue in the second part of the biosensor may be configured to be phosphorylated by the cell surface receptor when the cell surface receptor is activated. In particular, when their specific ligands bind to them, the cell surface receptors may undergo dimerization, thereby activating the receptor (for an RTK, this would activate their tyrosine kinase activity). This activation leads to the phosphorylation of tyrosine residues within the receptor itself (autophosphorylation) and also of downstream signaling molecules. In certain aspects, the second part includes pairs of tyrosine residues, such as pairs of residues located in immunoreceptor tyrosine-based activation motifs (ITAMs). For example, the ITAMs could include ITAMS from the CD3γ CD3δ, CD3ε, or CD3ζ chains of a T-cell receptor.

To produce the engineered biosensor, an engineered biosensor sequence (110) may be provided. The biosensor sequence (110) may include a first polynucleotide sequence (e.g., first part (111)) encoding a first part (101) of the biosensor (e.g., a cell surface receptor). The biosensor sequence may include a second polynucleotide sequence (e.g., second part (112)) fused to the first sequence, the second sequence encoding a second part (102) of the biosensor (e.g., a portion that includes at least a tyrosine residue that can be phosphorylated by the receptor). In certain aspects, repeats of the second sequence may be included. For example, there may be 2, 3, 4, 5, or 6 repeats of the second sequence. The engineered biosensor sequence (110) may include a third polynucleotide sequence (e.g., third part (113)) encoding a third part (103), such as a reporter tag (which may be e.g., a fluorescent tag).

In various aspects, a system may be provided. The system may include an engineered biosensor (100) as disclosed herein, and a reporter protein (120), sometimes referred to as a reporter protein.

The reporter protein (120) may include two parts.

The first part (121) may be configured to specifically interact with a phosphorylated tyrosine residue of the first biosensor. The first part may be a structurally conserved protein domain. The structurally conserved protein domain may be, e.g., a Src Homology 2 (SH2) domain. SH2 domains typically bind short phosphotyrosine-containing peptide motifs. Preferably, the first part is a tandem domain, such as a tandem SH2 domain.

The second part (122) may be, e.g., a fluorescent tag (or fluorescent protein), a protein domain that triggers an intracellular signaling response, or a combination thereof.

To produce the reporter protein, an engineered reporter protein sequence (130) may be provided. The reporter protein sequence (130) may include a first polynucleotide sequence (e.g., first part (131)) encoding the first part of the reporter protein (e.g., the structurally conserved protein domain, such as an SH2 domain). A second polynucleotide sequence (e.g., second part (132)) and/or an optional third polynucleotide sequence (e.g., third part (133)) may be fused to the first sequence. The second and/or optional third polynucleotide sequence may include a sequence encoding a reporter tag, such as a fluorescent tag. The second and/or optional third polynucleotide sequence may include a sequence encoding a protein domain that triggers an intracellular signaling response.

Examples of such protein domains include, e.g., an SH2 Domain (Src Homology 2), and SH3 Domain (Src Homology 3), a PH Domain (Pleckstrin Homology), a PDZ Domain (Post-synaptic Density 95, Drosophila disc large, and Zonula occludens-1), a BTK Domain (Bruton Tyrosine Kinase Domain), a Rho-GTPase Domain, a G-Protein Coupled Receptor (GPCR) Domains, and/or a Calcineurin Domain.

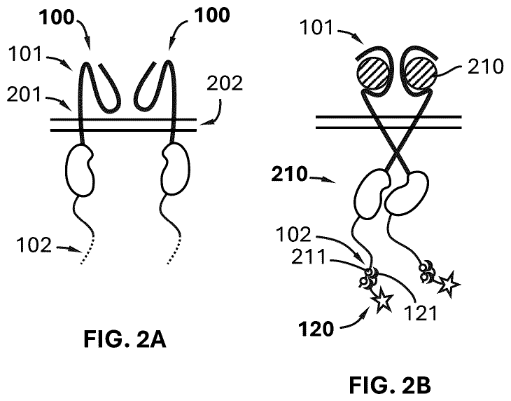

The system can be visualized by considering FIGS. 2A-2B. In FIG. 2A, which shows a state of low RTK activity, a portion (201) of the first part (101) of the biosensor (100) is shown as passing through a membrane (202). As shown in FIG. 2B, which shows a state of high RTK activity, such as in the presence of a target ligand (205), the first part (101) of each biosensor binds the target ligand, resulting in dimerized receptors (210), leading to the autophosphorylation of tyrosine residues (211) in the receptor tails (e.g., second part (102)). These tyrosine residues (211), when phosphorylated, can recruit the reporter protein (120)—e.g., a fluorescently labeled tandem SH2 domain—with high specificity.

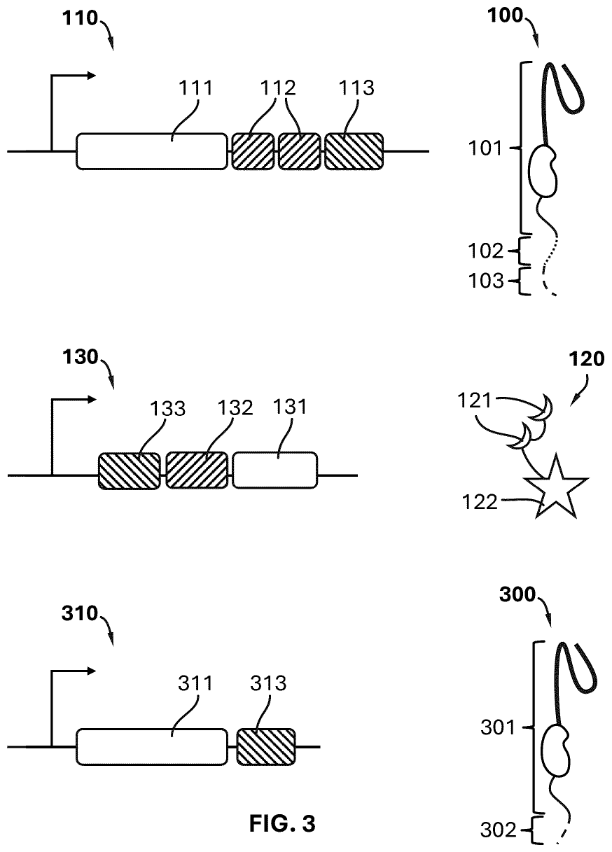

Embodiments of the system as described with respect to FIG. 1 can be used for single-target biosensors where the biosensors can homodimerize, but that will not work if the biosensor's RTK is ligandless. For example, ErbB2 has no known ligands. To allow such ligandless receptors to be utilized, a heterodimer approach may be used. Such an approach can be described with reference to FIG. 3.

In FIG. 3, one can see a similar arrangement to that of FIG. 1, but where a third component—the heterodimerizing partner protein (300) can be seen.

As one example, the first sequence (110) may include a first part (111) encoding ErbB2 fused to a second part (112) encoding CD3ε, and a third part (113) encoding a FusionRed fluorescent tag. The second sequence (130) may include a first part (131) coding ZtSH2, and a second part (132) encoding a near-infrared fluorescent protein (iRFP).

The dimerizing partner protein (300) may include a first part (301) and a second part (302), where the first part includes a cell surface receptor with at least one known ligand appropriate for the intended purpose (for the example here with the first sequence encodes ErbB2, this may be, e.g., EGFR), and the second part includes a reporter tag.

To produce the dimerizing partner protein, an engineered dimerizing partner protein sequence (310) may be provided. The dimerizing partner protein sequence (310) may include a first polynucleotide sequence (311) encoding the first part of the reporter protein (e.g., EGFR, etc.). A second polynucleotide sequence (312) may be fused to the first sequence, the second sequence encoding the second part of the biosensor (e.g., a reporter tag, such as a fluorescent tag).

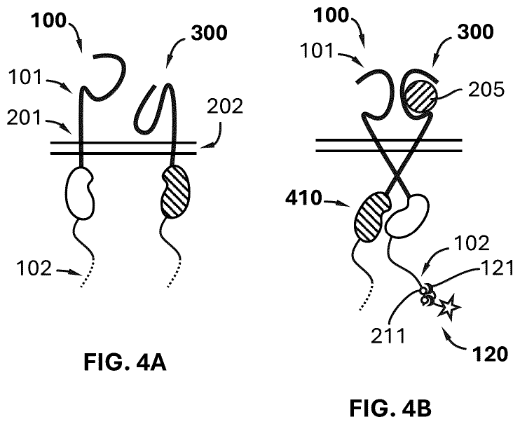

As seen in FIGS. 4A and 4B, in order to signal, the ligandless receptor (e.g., ErbB2) must heterodimerize with a ligand-binding member of the ErbB family (such as EGFR). The pYtag strategy enables measurements of ErbB2's activity despite the co-activation of epidermal growth factor receptor (EGFR). In FIG. 4A, showing a state of low RTK activity, a portion (201) of the first part (101) of the biosensor (100) is shown as passing through a membrane (202). As shown in FIG. 4B, which shows a state of high RTK activity, such as in the presence of a target ligand (205), the first part (101) the dimerizing partner protein (300) binds the target ligand, resulting in the biosensor (100) and its dimerizing partner protein (300) forming a heterodimer (410), leading to the autophosphorylation of tyrosine residues (211) in the receptor tails (e.g., second part (102)). These tyrosine residues (211), when phosphorylated, recruit the reporter protein (120)—e.g., a fluorescently labeled tandem SH2 domain—with high specificity.

The system may include a second engineered biosensor as disclosed herein, where the second biosensor may be orthogonal to the first biosensor. For example, the system may include one biosensor where the RTK is an EGFR, and another biosensor where the RTK is an insulin receptor.

In some embodiments, the orthogonal biosensors are in a system with two pairs of components, each pair akin to that seen in FIG. 1. For example, a first biosensor and first reporter protein could utilize a ZtSH2/CD3ε pair of proteins (e.g., reporter having ZtSH2, the biosensor having CD3ε), and a second biosensor and second reporter could utilize a VtSH2/SLP76 pair of proteins (e.g., reporter having VtSH2, the biosensor having SLP76. In some embodiments, the first biosensor and second biosensor could utilize the same RTK (e.g., EGFR). In other embodiments, the first biosensor and second biosensor could utilize different RTKS (e.g., the first biosensor could utilize ErbB2, the second biosensor could include EGFR)

In various aspects, a method for monitoring receptor tyrosine kinase activity may be provided. The method may include measuring a first luminescence of at least one cell, the at least one cell having a cell surface. The method may include allowing a cell surface receptor of an engineered biosensor as disclosed herein to interact with the cell surface of the at least one cell and activate, convert a tyrosine residue of the biosensor into a phosphorylated tyrosine residue, and recruit a reporter protein as disclosed herein to the phosphorylated tyrosine residue, where the reporter protein includes a fluorescent protein. The method may then include measuring a second luminescence of the at least one cell. As disclosed herein, the cell surface receptor may be, e.g., an EGFR, a FGFR, a PDGFR, a VEGFR, etc. The method may include continuously measuring luminescence or fluorescence of the at least one cell over a period of time. The method may include determining a difference in luminescence or fluorescence of the at least one cell, after exposing the at least one cell to a pharmaceutical agent.

The disclosed approach allows for a fusion between a gene encoding a type of cell surface receptor (receptor tyrosine kinase, or receptor that recruits tyrosine kinase activity) and a sequence encoding of a tyrosine residue that can be phosphorylated by the receptor.

Inter alia, the disclosed approach can be employed to screen for drugs that alter receptor tyrosine kinase activity, a major class of therapeutically relevant drug targets, as well as for other processes, e.g., drug screening—using this as a biosensor for monitoring how receptor activity is changed by small molecule drugs; and engineering new cell-based therapies by building “designer” receptor tyrosine kinases that can reprogram cell physiology.

The disclosed approach is understood to be the only technology that can report on the activity of any receptor tyrosine kinase of interest in living cells, and no other technology is understood to offer this combination of specificity and sensitivity. Also, because it comprises protein sequences, it can be used to design new receptors that program additional cellular responses, for example, to improve skin wound healing or nerve regeneration after injury.

The disclosed composition of matter is made by generating new gene sequences and introducing them into cells (e.g., using mRNA delivery, similar to the COVID vaccine, or using viral vectors).

### EXAMPLES

This biosensing approach was first applied to detect the activation of EGFR, the most well-characterized of the 58 known human RTKs. Six ITAMs from the CD3γ, CD3δ, CD3ε, and CD3ζ chains of the TCR. In each case, three identical repeats of the ITAM were fused to the C-terminal tail of EGFR followed by the FusionRed fluorescent protein (EGFR-ITAM-FusionRed). Each receptor was the co-expressed with an iRFP-labeled ZtSH2 (iRFP-ZtSH2) in NIH 3T3 fibroblasts, which express negligible levels of EGFR. In cells expressing EGFR-ITAM-FusionRed, treatment with epidermal growth factor (EGF) resulted in rapid translocation of ZtSH2 from the cytosol to the cell membrane, which was assessed by quantifying the percentage of ZtSH2 cleared from the cytosol. Clearance of ZtSH2 from the cytosol was quickly reversed by treatment with Gefitinib, an inhibitor of EGFR kinase activity, indicating that ZtSH2 can serve as a rapid, reversible reporter of EGFR activation. Cells lacking EGFR or expressing an ITAM-less EGFR-FusionRed exhibited no change in ZtSH2 localization in response to treatment with EGF or Gefitinib, demonstrating that the ZtSH2 reporter responds specifically to the activation of an ITAM-labeled RTK. Furthermore, a Grb2-based reporter localized to both EGFR-CD3ε-FusionRed and EGFR-FusionRed in cells stimulated with EGF, confirming that the Grb2-based reporter cannot discriminate between ITAM-labeled and ITAM-less RTKs

Although all six ITAMs that were tested appear capable of functioning as biosensors of RTK activity, the CD3ε ITAM was chosen to be focused on for all subsequent experiments due to its reported selectivity for ZtSH2 over other phosphotyrosine-binding domains. This two-component biosensor can be considered a pYtag: a tyrosine activation motif that recruits its complementary tSH2 reporter to report on signaling.

Since pYtags introduce additional tyrosine residues and SH2-containing peptides to an RTK signaling complex, it was considered whether this biosensing strategy interferes with signaling downstream of EGFR. NIH 3T3 cells expressing either EGFR-FusionRed or EGFR pYtag (EGFR-CD3ε-FusionRed; iRFP-ZtSH2) were stimulated with EGF and signaling responses were measured as a function of time by immunoblotting.

For all immunoblotting, cells were lysed in ice-cold rapid-immunoprecepitation (RIPA) buffer (1% Triton X-100, 50 mM HEPES, 150 mM NaCl, 1.5 mM MgCl₂, 1 mM extazic acid (EGTA), 100 mM NaF, 10 mM sodium pyrophosphate, 1 mM Na₃VO₄, 10% glycerol) supplemented with freshly prepared protease and phosphatase inhibitors. Protein levels were quantified using a PIERCE™ BCA protein assay kit (Thermo Fisher Scientific), before being mixed with 6× Laemmli buffer/2-mercaptoethanol, heated for 5 min at 95° C., and loaded onto a 4-12% Bis-Tris gel (Invitrogen) for electrophoresis. Gels were transferred to a nitrocellulose membrane using the iBlot dry transfer system (Thermo Fisher Scientific), blocked in TBST with 5% milk for 30 min at room temperature, and incubated in primary antibody overnight at 4° C. Before imaging, membranes were washed in TBST (mixture of tris-buffered saline, TBS, and polysorbate-20) and treated with either IRDYER 680CW or 800CW secondary antibodies (LI-COR) for 1 h. Imaging was performed with the LI-COR Odyssey Infrared Imaging System. Immunoblot images were analyzed using FIJI. The signal of the target protein was normalized by the signal of β-actin, which was used as a loading control. To normalize EGFR phosphorylation levels (pEGFR) to those of total EGFR, β-actin-normalized pEGFR signals were divided by their respective β-actin-normalized EGFR signals from the same experiment.

It was found that EGFR, Erk, and Akt were phosphorylated at similar levels in the two cell lines, suggesting that ITAM phosphorylation and ZtSH2 recruitment do not interfere with signaling downstream of EGFR.

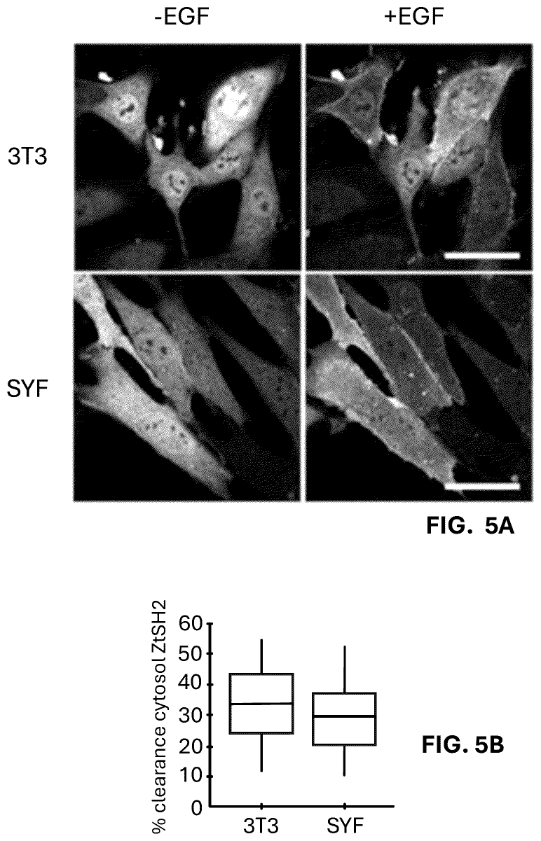

In T cells, CD3 ITAMs are typically phosphorylated by Src family kinases (SFKs) upon engagement of T-cell receptors (TCRs). Because EGFR also activates SFKs, it was reasoned that pYtags could be phosphorylated indirectly by EGFR-associated SFKs rather than by the kinase domain of EGFR itself. To test whether SFK-mediated phosphorylation is required for pYtag function, EGFR pYtag was expressed in mouse embryonic fibroblasts lacking all three ubiquitously expressed SFKs: Src, Yes, and Fyn (SYF cells). As in NIH 3T3 cells, SYF cells exhibited strong clearance of ZtSH2 from the cytosol after stimulation with EGF. See FIGS. 5A-5B. Combined with the rapid responses observed after EGFR stimulation and inhibition, these results suggest that the EGFR pYtag acts as a direct biosensor of EGFR activity.

### PYtags Reveal the Dynamics of EGFR Signaling at High Spatiotemporal Resolution

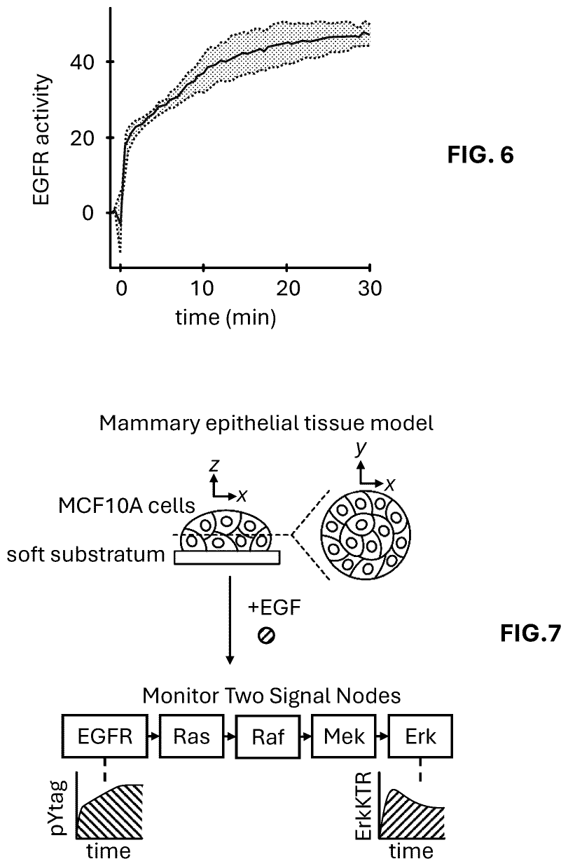

Because ZtSH2 can in principle be recruited rapidly on and off the membrane and may localize to distinct subcellular compartments, it was reasoned that pYtags could be used to monitor the subcellular activity of an RTK in individual cells over time. To test this possibility, rapid, high-resolution imaging of NIH 3T3 cells treated with EGF was first performed. It was found that ZtSH2 was cleared from the cytosol in a biphasic manner, with an initial phase of rapid cytosolic clearance occurring within the first ˜40 sec, followed by a further gradual increase in cytosolic clearance over the subsequent ˜20 min. See FIG. 6. Similar biphasic responses for EGFR were observed previously at the population-level using a split luciferase system and in vitro phosphorylation assays, but have never been reported for individual cells.

Over longer time scales, EGFR can be trafficked through internalization, degradation, and recycling back to the cell membrane. Notably, it was observed that pYtag-expressing cells stimulated for at least 30 min with EGF contained internalized vesicles that were positive for both total EGFR and ZtSH2. Subsequent treatment with Gefitinib eliminated ZtSH2 from EGFR-positive vesicles within minutes, suggesting that the enrichment of ZtSH2 at vesicles is indicative of signaling from endosomal compartments. These results are consistent with prior reports that internalized EGFR remains bound to its ligand and can transmit signals from endosomal compartments.

At the tissue scale, the spatiotemporal distribution of RTK signaling can also be regulated by several processes including paracrine signaling, morphogen gradients, and the mechanical properties of the local microenvironment. It was therefore asked whether pYtags could be used to monitor RTK signaling in multicellular contexts. It was previously found that MCF10A human mammary epithelial cells form multicellular clusters when cultured on soft substrata and exhibit a complex spatial pattern of EGF binding at cell membranes.

To prepare polyacrylamide substrata, 1.5-mm-thick glass coverslips were pre-treated with glutaraldehyde. First, coverslips were treated with 0.1 N NaOH for 30 min, followed by rinsing with deionized water and air drying. Coverslips were then treated with 2% aminopropyltrimethoxysilane (Sigma Aldrich) in acetone for 30 min, washed three times with acetone, and left to air dry. Coverslips were treated with 0.5% glutaraldehyde (Sigma Aldrich) in PBS for 30 min, washed with deionized water, and left to air dry. Custom glass-bottom dishes were prepared by replacing the bottoms of 35 mm TCPS dishes with glutaraldehyde-treated coverslips, which were sealed using PDMS (Sigma Aldrich).

Measurements from fixed tissues revealed that EGF binds rapidly to the media-exposed membranes on the surface of the cluster but is excluded from lateral membranes and from cells located within the interior of the cluster. To investigate the dynamics of this spatial pattern, EGFR signaling was monitored using two live-cell biosensors: EGFR pYtag and a kinase translocation reporter for downstream signaling through Erk (ErkKTR). See FIG. 7.

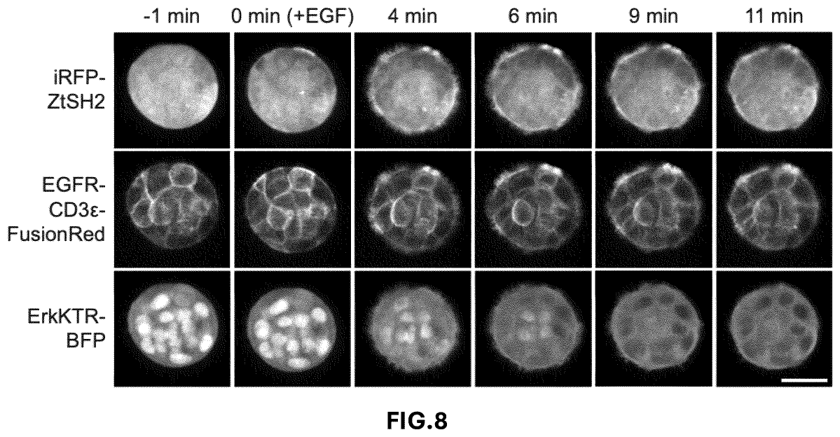

MCF10A cells cultured on soft substrata were treated with EGF, and a radially directed wave of EGFR and Erk signaling was observed: activation first appeared in cells at the periphery of the clusters before appearing in cells at the interior. See FIG. 8. In cells at the periphery of clusters, it was found that ZtSH2 was first enriched at the media-exposed membrane before localizing to lateral membranes, highlighting differences in receptor-level signaling between membrane sub-compartments. Quantifying EGFR and Erk responses confirmed the qualitative observations, revealing a 2-4 min delay in EGFR and Erk signaling between the periphery and interior of clusters. Notably, Erk responses also trailed those of EGFR by ˜4 min, consistent with the delay in signal transmission previously observed from Ras to Erk. These data support a model in which ligand-receptor interactions are limited at cell-cell contacts, producing an inward-traveling wave of RTK activation and downstream signaling. More broadly, our data demonstrate that pYtags can be used to reveal RTK signaling dynamics at seconds-scale resolution and over both subcellular and multicellular length scales.

### PYtags Distinguish Ligand- and Dose-Dependent Signaling Dynamics

The EGFR pYtag was next applied to study an unresolved feature of RTK signaling dynamics. While it has long been known that different RTKs regulate cellular processes by altering signaling dynamics, recent evidence suggests that different ligands alter signaling dynamics through the same receptor. For instance, high-affinity EGFR ligands such as EGF produce long-lived EGFR dimers that are internalized and degraded, whereas low-affinity ligands such as epiregulin (EREG) and epigen (EPGN) induce shorter-lived EGFR dimers that prolong signaling, leading to distinct outcomes in cell fate. How these different ligands might alter minutes-timescale EGFR signaling dynamics in single cells remains unknown.

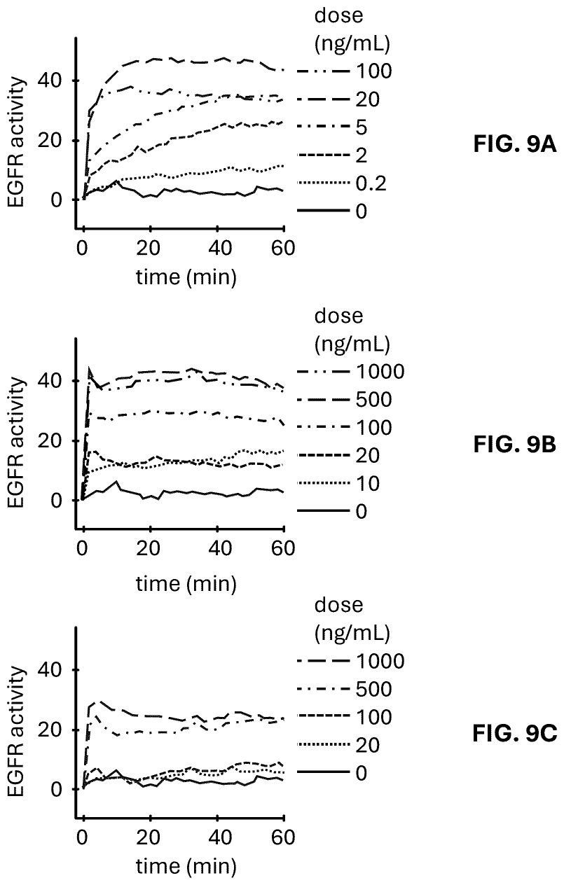

To answer this question, EGFR pYtag-expressing NIH 3T3 cells were treated with varying doses of either EGF or one of two low-affinity ligands, EREG or EPGN. It was found that the kinetics of EGFR activation varied substantially with the dose of EGF, from a gradual rise in activity at lower doses (0.2-2 ng/mL) to a more rapid, biphasic response at higher doses (5-100 ng/ml). See FIG. 9A. In contrast, treatment with the low-affinity ligands EREG and EPGN led to a rapid rise in EGFR activity followed by a plateau within minutes. See FIGS. 9B (EREG) and 9C (EPGN). A small but reproducible transient peak of EGFR activation was observed shortly after treatment with EREG and EPGN but not with EGF, suggestive of weak negative feedback after stimulation with low-affinity ligands (see FIGS. 9A-9C). These results demonstrate that different ligands produce profoundly different signaling responses in the minutes immediately after EGFR stimulation. They also reveal an unusual property of the low-affinity RTK ligands EREG and EPGN: altering their concentrations has a strong effect on the amplitude, but not the kinetics, of EGFR activation.

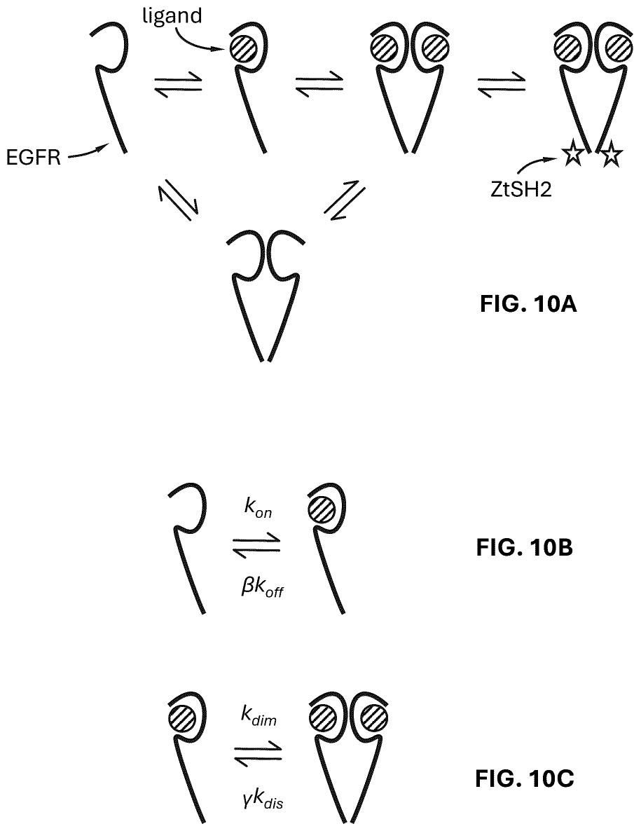

We constructed a mathematical model of the EGFR pYtag based on mass-action kinetics to better understand these ligand-dependent EGFR signaling responses. This is schematically shown in FIG. 10A. In the model, ligand-free EGFR monomers bind to ligands, dimerize, undergo autophosphorylation and, lastly, recruit, e.g., ZtSH2 to ligand-bound dimers. EGFR dimerization in the absence of ligand were also modeled, based on several observations of these inactive dimers. In the model, ligand-free dimers may also bind to ligand and recruit ZtSH2, bypassing the intermediate step of transitioning from monomer to dimer. Notably, it was found that ligand-free EGFR dimers were necessary to reproduce the biphasic signaling dynamics observed after stimulation with EGF. It was assumed that different EGFR ligands could alter two rate constants in the model (see FIGS. 10B-10C). First, high- and low-affinity ligands are expected to bind to their receptor with different affinities, which were modeled by varying the dissociation rate of ligand-receptor complexes using a scaling parameter β. See FIG. 10B. Second, low-affinity ligands have been reported to produce structurally different EGFR dimers compared to high-affinity ligands, thereby reducing the dimerization affinity of ligand-bound receptors; this dimerization affinity was modeled by varying the dissociation rate of ligand-bound dimers using a scaling parameter γ (see FIG. 10C).

To define which ligand-specific interactions correlate with differential signaling dynamics (see FIGS. 9A-9C), β and γ were systematically varied while simultaneously varying ligand doses. Setting both β and γ to 1 reproduced the dynamics of EGFR signaling in response to EGF (see FIG. 9A). Ligand binding affinity was then decreased by increasing β, which increased the dose of ligand required for activation but failed to qualitatively alter the shape of the response curves as we had observed experimentally. In contrast, decreasing the dimerization affinity of ligand-bound receptors by increasing γ was sufficient to reproduce many of the features that varied with ligand identity. At low values of γ, an EGF-like response was observed consisting of a biphasic and gradual approach to steady-state, due to the initial activation of ligand-free dimers, and subsequent dimerization and activation of the remaining monomeric receptors. In contrast, high values of γ produced an EREG/EPGN-like initial peak of receptor activation followed by a plateau (see FIGS. 9B-9C), due to rapid activation of ligand-free dimers but less additional dimerization of ligand-bound monomeric receptors. In sum, the model predicts that the EGFR signaling dynamics produced by high- and low-affinity ligands arise from altered dimerization affinities of ligand-bound receptors.

To validate the prediction of our model, the dimerization affinity of EGFR was experimentally altered and signaling responses were monitored using pYtags. One can turn to glioblastoma multiform (GBM)-associated mutations in the extracellular domain of EGFR (R84K and A265V point mutations), which were previously shown to increase the dimerization affinity of EREG- and EPGN-bound receptors. These GBM-associated mutations were first modeled in EGFR by decreasing β and γ by 6-fold and 650-fold, respectively. These modifications led to the prediction that 20 ng/mL EREG would induce a stronger, more gradual signaling response in GBM-associated mutants compared to the wild-type receptor (see FIG. 10A). NIH 3T3 cell lines expressing pYtag biosensors of EGFR variants harboring either the R84K or A265V mutation were then generated.

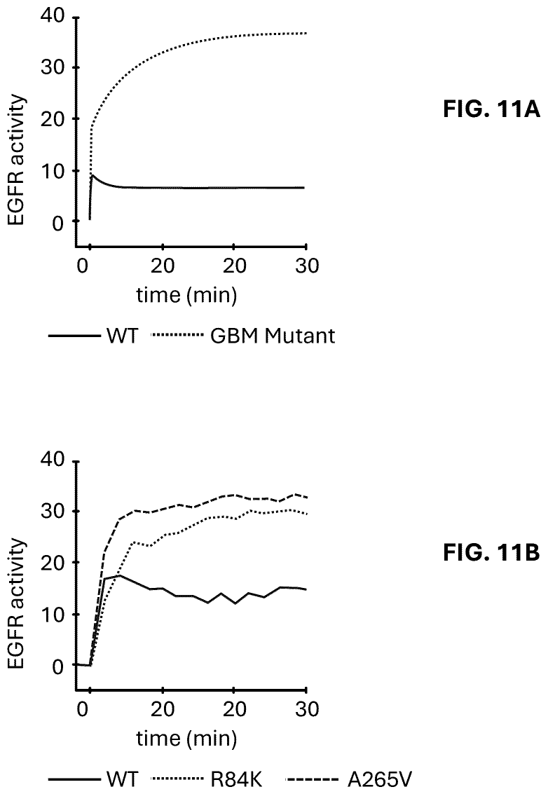

Treating these cells with 20 ng/ml EREG induced signaling responses that closely matched the predictions of the model: cells expressing WT EGFR exhibited a transient peak of activation and rapid plateau, while cells expressing GBM-associated mutants exhibited a stronger response that gradually increased over time. See FIGS. 11A-11B. The data reveal ligand-dependent EGFR signaling dynamics at unprecedented temporal resolution and point to the dimerization affinity of ligand-bound receptors as a key parameter that governs these responses. More broadly, combining pYtags with mathematical modeling can shed mechanistic insight into the signaling dynamics of RTKs.

### PYtags Report the Activity of Distinct RTKs in Heterodimeric Complexes

One advantage of the pYtag approach is its modularity: ITAMs can in principle be fused to the C-termini of many different RTKs and recruit ZtSH2 upon stimulation. Therefore, it was tested whether pYtags could be adapted to monitor the activity of receptors other than EGFR. pYtag biosensors were designed for two additional RTKs: 1) ErbB2, a ligandless member of the ErbB family that signals by heterodimerizing with ligand-bound EGFR; and 2) fibroblast growth factor receptor 1 (FGFR1), a non-ErbB family RTK. ErbB2 is a particularly challenging RTK to study because it can only be activated in conjunction with an additional RTK (e.g., EGFR), so Grb2-based biosensors would be unable to distinguish between the activity of EGFR and ErbB2.

Starting from NIH 3T3 cells expressing iRFP-ZtSH2, cell lines expressing either an ErbB2 pYtag (ErbB2-CD3ε-FusionRed), an FGFR1 pYtag (FGFR1-CD3ε-FusionRed), or ITAM-less variants of each were generated. For the ErbB2 case, cells with EGFR-Citrine were further transduced to express both required components of a functional receptor heterodimer (see FIG. 3, and FIGS. 4A-4B).

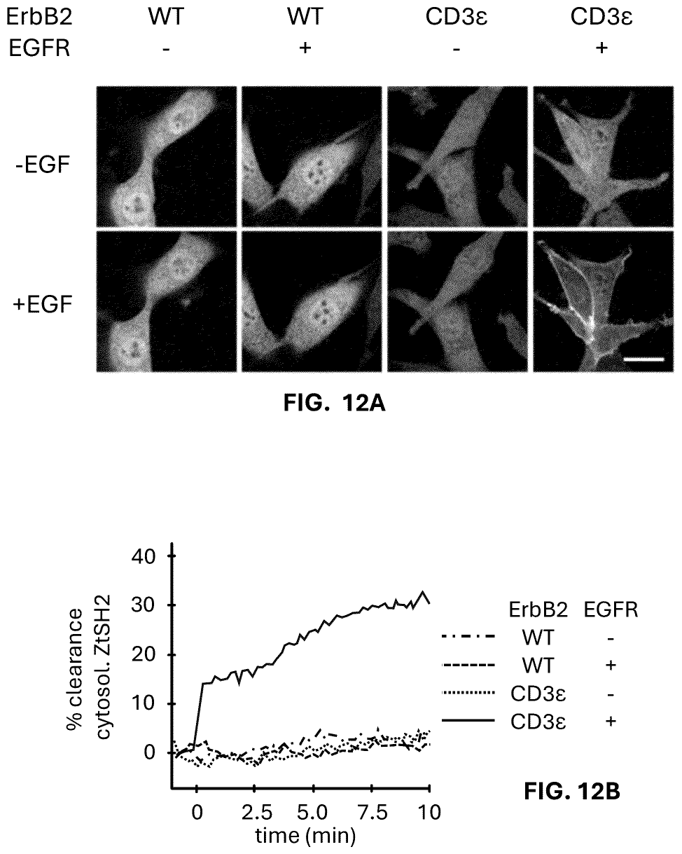

It was found that pYtags generalized well to both ErbB2 and FGFR1. As with the EGFR pYtag, the responses of the ErbB2 and FGFR1 pYtags were quantified by measuring the clearance of ZtSH2 from the cytosol in response to treatment with activating ligands. Stimulating ErbB2 pYtag-expressing cells with EGF led to clearance of ZtSH2 from the cytosol, a response that required both the presence of ITAMs on ErbB2 and co-expression of EGFR (FIGS. 12A and 12B). Similarly, stimulating FGFR1 pYtag-expressing cells with FGF4 induced cytosolic clearance of ZtSH2, which did not take place in cells that expressed an ITAM-less FGFR1. Both FGFR1-FusionRed and FGFR1-CD3ε-FusionRed were also prominently clustered in internal compartments that recruited ZtSH2 in an ITAM-dependent manner, suggesting a high degree of basal signaling from these internal compartments that could be due to ectopic expression of the receptor. The pYtag approach is indeed modular, and this strategy can be readily adapted for monitoring the activation of distinct RTKs in individual cells.

### Multiplexing RTK Biosensors Using Orthogonal pYtags

The disclosed pYtag design strategy relies on the high selectivity that can be produced by multivalent association between pairs of phosphotyrosine motifs and tSH2 domains. It can thus be understood that two orthogonal pYtags could be deployed to monitor the activation of distinct RTKs in the same cell. To this end, an example was created taking advantage of another phosphotyrosine/tSH2 interaction pair from immune-specific signaling proteins to build a pYtag that functions orthogonally to the above-described CD3ε/ZtSH2 system. Following its binding to phosphorylated ITAMs on the TCR, ZAP70 phosphorylates non-ITAM tyrosine residues on the scaffold protein SLP76, which subsequently recruit multiple signaling components via SH2-mediated interactions. Because immune signaling requires these events to be discreet, sequential, and to operate in proximity to each other, ZtSH2- and SLP76-recruited SH2s must have orthogonal binding specificities. This feature can be exploited to engineer a second pYtag. Here, two tyrosine residues of SLP76 were identified, Y128 and Y145, which when phosphorylated bind to SH2 domains from the guanine nucleotide exchange factor Vav and the kinase ITK, respectively. To create a synthetic tSH2 that could bind tightly to the Y128/Y145 motif, the SH2 domains of Vav and ITK were fused together with a 10 bp glycine-serine linker to create VISH2. The suitability of the SLP76/VISH2 system was then characterized for monitoring RTK activity and quantified its crosstalk with the CD3ε/ZtSH2 system.

To confirm whether the SLP76/VISH2 system functions as a pYtag biosensor in an orthogonal manner to CD3ε/ZtSH2, NIH 3T3 cells were generated that stably co-expressed Clover-VISH2 (e.g., a VISH2 reporter) and iRFP-ZtSH2 (e.g., a ZtSH2 reporter). These cells were then further transduced with a variant of EGFR labeled with either three repeats of the E123-E153 region of SLP76 (EGFR-SLP76-BFP) or three repeats of the CD3ε ITAM (EGFR-CD3ε-BFP). Stimulating the two cell lines with EGF and monitoring the localization of VISH2 and ZtSH2 revealed that cells expressing SLP76-labeled EGFR exhibited clearance of VISH2 from the cytosol but no crosstalk with ZtSH2. Conversely, cells expressing CD3ε-labeled EGFR exhibited strong clearance of ZtSH2 from the cytosol with minimal crosstalk with VISH2. The VISH2/SLP76 and CD3ε/ZtSH2 pYtags thus operate independently of one another to report the activity of EGFR.

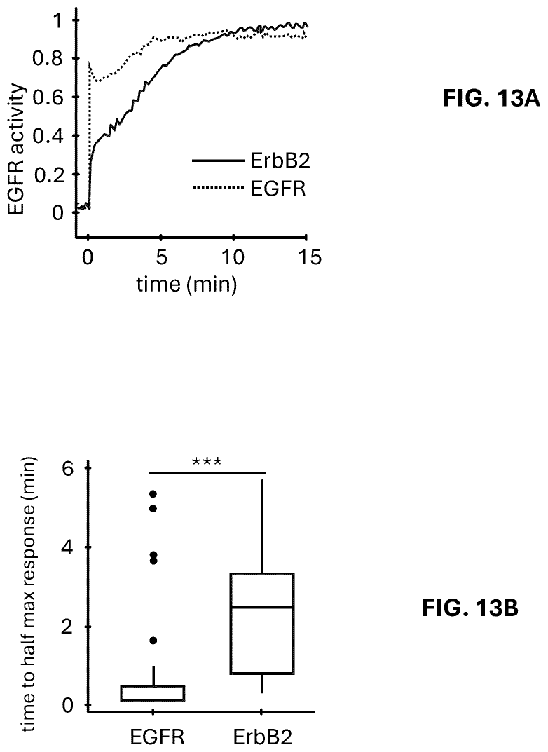

Having characterized an orthogonal pair of pYtags, it was recognized that these biosensors could be used to simultaneously monitor the activation of both EGFR and ErbB2. NIH 3T3 cells that co-expressed pYtags for EGFR (Clover-VISH2; EGFR-SLP76-BFP) and ErbB2 (iRFP-ZtSH2; ErbB2-CD3ε-FusionRed) were generated. Cells were treated with 100 ng/mL EGF and it was observed that both VISH2 and ZtSH2 reporters cleared from the cytosol, as would be expected from the activation of EGFR and ErbB2 heterodimers. However, the biosensors revealed markedly different dynamics of receptor activation. See FIGS. 13A-13B. VISH2 cleared from the cytosol almost immediately (<30 sec), indicating rapid phosphorylation of the EGFR C-terminal tail. In contrast, ZtSH2 exhibited a more gradual clearance from the cytosol (˜3 min), suggesting a delay in ErbB2 phosphorylation. To verify that these dynamics are not due to the identity of the pYtag used to monitor each receptor or an artifact of the cell line expressing multiplexed biosensors, the dynamics of EGFR and ErbB2 activation were compared across all experiments that used 100 ng/mL EGF, regardless of the cell line or pYtag used. Although the absolute magnitude of tSH2 biosensor clearance varied between cell lines (likely due to differences in affinity and expression level), the dynamics of EGFR and ErbB2 activation were highly reproducible in all cases, irrespective of the cell line or pYtag. These data demonstrate that pYtag measurements faithfully reflect the activation of the RTK on which they report. These results suggest that EGFR and ErbB2 are activated in distinct phases: EGFR is activated first, within seconds of stimulation, and then both EGFR and ErbB2 are further activated over the subsequent minutes.

### PYtags Can Monitor the Activity of Endogenous RTKs

One drawback to the pYtag approach is its reliance on ectopically expressing an ITAM-labeled receptor of interest. Increasing the expression levels of RTKs can alter signaling dynamics, downstream pathway engagement, and cell fate outcomes. Conversely, the low expression levels of endogenous RTKs may only drive weak recruitment of tSH2, making biosensing by live-cell microscopy difficult. It was therefore considered whether pYtags could be adapted to monitor the activity of endogenously expressed RTKs.

CRISPR/Cas9-based genome editing was used to label the C-terminus of endogenous EGFR with three repeats of the CD3ε ITAM and a fluorescent protein via homology-directed repair (HDR) in HEK 293T cells.

HEK 293T cells with CD3ε-mNeonGreen inserted at the endogenous EGFR locus were generated by transfecting cells with 1) a pX330 plasmid containing a human codon-optimized SpCas9 and guide RNA targeting EGFR (pX330 EGFR-sgRNA) and 2) a homology-directed repair template comprised of three repeats of the CD3ε ITAM and mNeonGreen flanked by 800 bp of EGFR homology regions (pUC19 EGFRup-CD3ε-mNeonGreen-EGFRdown) (Ran et al., 2013). HEK 293T cells were first seeded at 130,000 cells/well in a 24-well plate. 24 h later, cells were transfected with 330 ng pX330 EGFR-sgRNA and 170 ng pUC19 EGFRup-CD3ε-mNeonGreen-EGFRdown using LIPOFECTAMINE™ 3000 transfection reagent (Thermo Fisher Scientific) and left to culture for another 48 h. Clonal cell lines were then isolated using fluorescence-activated cell sorting on a Sony SH800S cell sorter, and localization of mNeonGreen was assessed by confocal microscopy. A candidate clonal cell line exhibiting membrane-localized mNeonGreen was then used for further validation.

To verify genomic integration of CD3ε-mNeonGreen, genomic DNA was isolated from parental and knock-in 293T cells using a PURELINK™ Genomic DNA Mini Kit (Invitrogen). PCR using HiFi polymerase was then used to amplify a region of genomic DNA using primers specific to homology regions flanking the CD3ε-mNeonGreen insertion. PCR products were then run through an agarose gel by electrophoresis and imaged using an Axygen Gel Documentation System. In the gel, endogenous EGFR was expected to appear as a ˜1.2 kb product, while EGFR-CD3ε-mNeonGreen was expected to appear upshifted as a ˜2.1 kb product. The expression of EGFR-CD3ε-mNeonGreen protein was verified by immunoblotting.

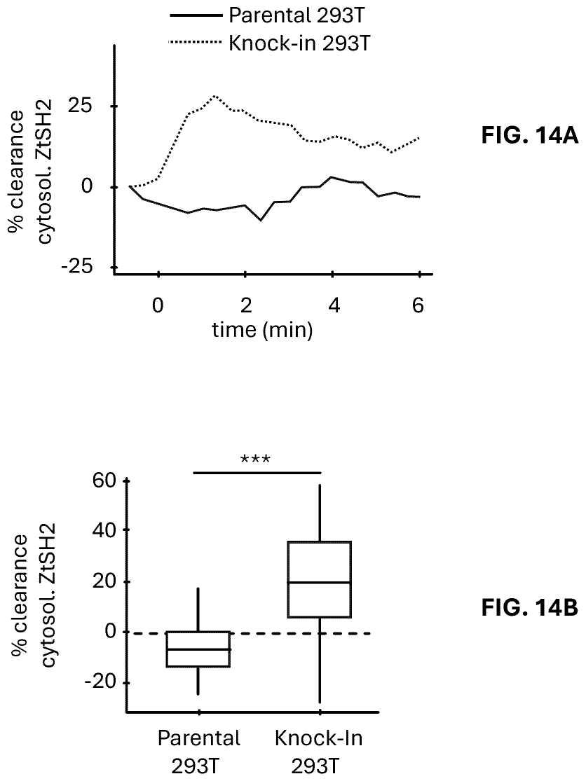

mNeonGreen was chosen as the fluorescent protein label because of its exceptional brightness, which aids detection at low levels of endogenous expression. An mNeonGreen-expressing clonal cell line was sorted and proper labeling of EGFR with CD3ε-mNeonGreen was confirmed by PCR of genomic DNA and immunoblotting. Both parental 293T and EGFR-CD3ε-mNeonGreen-expressing (knock-in 293T) cells were then transduced with an mScarlet-labeled ZtSH2 to take advantage of this fluorescent protein's high brightness for detecting subcellular ZtSH2 localization even at low expression levels. ZtSH2 localization was then monitored after treatment with EGF in both parental and knock-in cells. Knock-in 293T cells exhibited rapid clearance of ZtSH2 from the cytosol following EGF treatment, whereas no ZtSH2 response was observed in parental cells. See FIGS. 14A-14B. Striking differences were observed in the dynamics of endogenous receptor activation compared to experiments in which EGFR was ectopically expressed: endogenous EGFR exhibited a decrease in activity within minutes after EGF stimulation whereas membrane recruitment was sustained for at least 60 min for ectopically expressed EGFR (see FIGS. 9A-9C). Rapid and near-complete internalization of endogenous EGFR was also observed from the cell membrane, with some internalized vesicles retaining residual ZtSH2 labeling. It was therefore concluded that pYtags can be used to monitor the activation state of endogenously expressed RTKs, opening the door to single-cell studies of RTK signaling dynamics in minimally perturbed contexts.

Therefore, disclosed herein are biosensors, referred to as pYtags, which provide a biosensing strategy for monitoring the activity of a specific RTK in living cells. pYtags rely on tyrosine activation motifs that are selectively bound by corresponding domains (preferably tSH2 domains) to minimize interactions with endogenous phosphotyrosine motifs and SH2 domains. This design principle confers selectivity to certain steps in T cell signaling, such as the recruitment of the kinase ZAP70 to phosphorylated ITAMs within activated TCRs. This property of immune signaling proteins may be exploited to build two orthogonal pYtags: for example, a first pYtag based on ITAMs from the TCR and the tSH2 domain of ZAP70, and a second pYtag based on the tyrosine-containing scaffold protein SLP76 and its SH2-containing binding partners Vav and ITK. It is disclosed that pYtags can be applied to multiple RTKs (for example, EGFR, ErbB2, and FGFR1), providing a robust strategy to monitor receptor-level signaling in living cells.

pYtags quantitatively report on RTK activity with seconds-scale precision and can be applied to monitor signaling at both subcellular and multicellular length scales. Responses are highly reproducible across multiple experiments and cell lines, even when the same receptor is monitored using different pYtag variants. This quantitative precision enables one to identify differences in EGFR signaling dynamics induced by high- or low-affinity ligands, and to observe distinct signaling dynamics for two ErbB-family receptors. pYtag measurements may be used to inform a mathematical model that, along with subsequent experimental validation, suggests that the dimerization affinity of ligand-bound receptors controls RTK signaling dynamics. The data suggest that pYtags represent a broadly applicable biosensing strategy to monitor the activity of any RTK of interest and can reveal new mechanistic insights for even the most well-studied RTKs.

pYtags have several advantages over previously reported biosensors of RTK activity. First, pYtags are modular: they can be applied to multiple RTKs without modification, unlike approaches that seek to identify phosphotyrosine/SH2 interactions present on endogenous RTKs. Second, pYtags are specific, reporting only on the activity of the RTK labeled by the tyrosine activation motif. Third, pYtags can be multiplexed: pYtags require one fewer fluorescent protein compared to FRET-based biosensors, and orthogonal variants of pYtags can be used to monitor distinct RTKs in the same cell. The human genome encodes at least 58 RTKs, most of which remain poorly characterized with respect to their spatial organization and signaling dynamics. Multiplexing pYtags opens the door to systematically characterizing how different combinations of RTKs and cognate ligands influence signal processing, as has been performed for the bone morphogenetic protein receptor family.

As disclosed herein, for the examples discussed, plasmids were constructed as follows. All constructs were cloned into the pHR lentiviral expression plasmid using inFusion cloning. Linear DNA fragments were produced by PCR using High Fidelity polymerase (Takara Bio), followed by treatment with DpnI to remove template DNA. PCR products were then isolated through gel electrophoresis and purified using the NUCLEOSPIN® gel purification kit (Takara Bio). Linear DNA fragments were then ligated using inFusion assembly and amplified in Stellar competent E. coli (Takara Bio). Plasmids were purified by miniprep (Qiagen) and verified by either Sanger sequencing (Genewiz) or whole-plasmid sequencing (Plasmidsaurus).

As disclosed herein, example cell lines were generated as follows. Constructs were stably expressed in cells using lentiviral transduction. First, lentivirus was produced by co-transfecting HEK 293T LX cells with pCMV-dR8.91, pMD2.G, and the expression plasmid of interest. 48 h later, viral supernatants were collected and passed through a 0.45 μm filter. Cells were seeded at ˜30% confluency and transduced with lentivirus 24 h later. 24 h post-seeding, culture medium was replaced with medium containing 10 μg/mL polybrene and 200-300 μL viral supernatant was added to cells. Cells were then cultured in virus-containing medium for 48 h. Populations of cells co-expressing each construct were isolated using fluorescence-activated cell sorting on a Sony SH800S cell sorter. Bulk-sorted populations were collected for all experiments, except for those using MCF10A cells and CRISPR/Cas9-edited HEK 293T cells, for which clonal lines were generated.

For the examples disclosed herein, cells were cultured as follows. NIH 3T3 cells, HEK 293T cells, and SYF cells were cultured in Dulbecco's Modified Eagle Medium (DMEM) (Gibco) supplemented with 10% fetal bovine serum (R&D Systems), 1% L-glutamine (Gibco), and 1% penicillin/streptomycin (Gibco). MCF10A-5E cells (Janes et al., 2010) were cultured in DMEM F12 (Gibco) supplemented with 5% horse serum (ATCC), 20 ng/mL EGF (R&D Systems), 0.5 μg/mL hydrocortisone (Corning), 100 ng/mL cholera toxin (Sigma-Aldrich), 10 μg/mL insulin (Sigma-Aldrich), and 1% penicillin/streptomycin (Gibco). All cells were maintained at 37° C. and 5% CO₂.

What is claimed:

1. An engineered biosensor for monitoring receptor activity, comprising:
a cell surface receptor; and
a tyrosine residue coupled to the cell surface receptor, the tyrosine residue configured to be phosphorylated by the cell surface receptor when the cell surface receptor is activated.

2. The engineered biosensor of claim 1, wherein the cell surface receptor is a receptor tyrosine kinase.

3. The engineered biosensor of claim 2, wherein the receptor tyrosine kinase is an epidermal growth factor receptor, a fibroblast growth factor receptor (FGFR), a platelet-derived growth factor receptor (PDGFR), or a vascular endothelial growth factor receptor (VEGFR).

4. A system, comprising:
a first engineered biosensor of claim 1; and
a second protein comprising:
a first part that specifically interacts with a phosphorylated tyrosine residue of the first engineered biosensor; and
a second part that is a fluorescent protein, a protein domain that triggers an intracellular signaling response, or a combination thereof.

5. The system of claim 4, wherein the first part is a structurally conserved protein domain.

6. The system of claim 5, wherein the structurally conserved protein domain is a Src Homology 2 (SH2) domain.

7. The system of claim 5, further comprising a second engineered biosensor of claim 1, the second engineered biosensor being orthogonal to the first engineered biosensor.

8. An engineered biosensor sequence, comprising:
a first polynucleotide sequence encoding a cell surface receptor; and
a second polynucleotide sequence fused to the first polynucleotide sequence, the second polynucleotide sequence encoding a tyrosine residue that can be phosphorylated by the cell surface receptor.

9. A method for monitoring receptor tyrosine kinase activity, comprising:
measuring a first luminescence of at least one cell, the at least one cell having a cell surface;
allowing a cell surface receptor of an engineered biosensor of claim 1 to interact with the cell surface of the at least one cell and activate, convert the tyrosine residue into a phosphorylated tyrosine residue, and recruit a reporter protein to the phosphorylated tyrosine residue, where the reporter protein includes a fluorescent protein; and
measuring a second luminescence of the at least one cell.

10. The method of claim 9, wherein the cell surface receptor is an epidermal growth factor receptor, a fibroblast growth factor receptor, a platelet-derived growth factor receptor, or a vascular endothelial growth factor receptor.

11. The method of claim 9, further comprising continuously measuring luminescence or fluorescence of the at least one cell over a period of time.

12. The method of claim 9, further comprising determining a difference in luminescence or fluorescence of the at least one cell, after exposing the at least one cell to a pharmaceutical agent.

13. The method of claim 9, wherein the reporter protein comprises a structurally conserved protein domain fused to the fluorescent protein.

14. The method of claim 13, wherein the structurally conserved protein domain is a Src Homology 2 (SH2) domain.

15. The method of claim 13, wherein the structurally conserved protein domain is fused to the fluorescent protein and a protein domain that triggers an intracellular signaling response.
